(19) United States
(12) Patent Application Publication
(10) Pub. No.: US 2017/0196834 A1
(43) Pub. Date: Jul. 13, 2017

(54) PREPARATION AND APPLICATION OF FLAVONOL AS BRAIN-TARGETING SYNERGIST

(71) Applicant: GUANGDONG PHARMACEUTICAL UNIVERSITY, Guangzhou, Guangdong (CN)

(72) Inventors: Weijian BEI, Guangzhou, Guangdong (CN); Jiao GUO, Guangzhou, Guangdong (CN)

(21) Appl. No.: 15/314,030

(22) PCT Filed: Dec. 30, 2015
(86) PCT No.: PCT/CN2015/099814

§ 371 Date: Nov. 25, 2016

(30) Foreign Application Priority Data
Dec. 31, 2014 (CN) 201410854987.3

(51) Int. Cl. A61K 31/352; A61K 31/7048; A61K 31/05; A61K 31/704

(52) U.S. Cl. CPC A61K 31/352; A61K 31/704; A61K 31/7048; A61K 31/05

(57) ABSTRACT

The present invention discloses a preparation and application of flavonol as a brain-targeting synergist. After long-term repeated studies, the inventors have found that some flavonol compounds, particularly kaempferide, rutin, troxerutin, myricetin, hesperidin, and hydroxy derivatives thereof, in particular their glycoside, ester, ether derivatives, can promote the drug molecules that have therapeutic or healthcare effect, such as ginsenoside, stilbene glucoside, resveratrol, levodopa, edaravone, vinpocetine, nicergoline, citicoline, oxiracetam, to enter brain tissues, to dramatically enhance drugs concentrations in the brain tissues and effective enhance the efficacy of drugs without increasing the plasma concentration.

### FIELD OF THE INVENTION

The present invention relates to new application of a chemical compound and a composition based on the application.

### BACKGROUND

The brain is the center of life, and its complex structure and function of the nervous system have become the most valuable research projects in the history of human science, which is receiving increasing attentions from scientists around the world.

Cerebral blood flow is the fastest, but many drugs enter the brain tissue after systemic administration much more slowly than entering the other tissues, thus, the concept of blood-brain barrier is proposed. The blood-brain barrier refers to blood-brain interstitial fluid and blood-cerebrospinal fluid barrier, which is composed of three barriers: blood-brain barrier (BBB), cerebro-spinal fluid-brain barrier and blood-cerebro-spinal fluid barrier. The function of blood-brain barrier is to selectively block some substances from entering the brain tissues. Since the surface area of the blood-brain barrier is about 5000 times of the surface area of the blood-cerebrospinal fluid barrier, therefore, the blood-brain barrier is the main barrier to control the endogenous and exogenous substances from entering the brain parenchyma, which contributes to maintain the stability of brain tissue environment.

Ischemic stroke, stroke sequelae, neurodegenerative diseases and other brain diseases are major threats to the health of the elderly. With the acceleration of population aging and the increasing incidence of mental illnesses, cerebrovascular diseases (cerebral ischemic injury-related diseases, including stroke, stroke sequelae, vascular dementia), neurodegenerative diseases (such as Parkinson's disease (PD), Alzheimer's disease (AD), senile dementia, etc.) and mental diseases (also called psychiatric disorders), epilepsy and other brain nerve and mental diseases have brought heavy burden on patients, families and society. However, due to the presence of blood-brain barrier (BBB), most drugs are restricted to enter the brain or cannot reach the required effective concentration for treatment, which greatly limits the treatment of brain diseases^{[1-4]}.

Traditional Chinese medicines have been used for the prevention and treatment of cardiovascular and cerebrovascular diseases for a long time, with significant effects and advantages such as safety, low toxicity and less side effects, etc.^{[6, 7]}. For example, ginsenoside extract, stilbene glucoside extract of Radix polygoni multiflori are important natural drugs for the treatment of brain diseases, however, their active ingredients ginsenoside Rb1, Rd, Re, Rg1, stilbene glucoside, resveratrol, and chemical drugs levodopa, edaravone, vinpocetine, nicergoline, citicoline, oxiracetam are not easy to penetrate the BBB to the brain tissues to function.

Therefore, resolving the problem of drugs penetrating the BBB has become a key and hot issue for the treatment of brain diseases^{[1, 3, 5]}.

### SUMMARY

The object of the invention is to provide an application of flavonol as a brain-targeting drug synergist.

Another object of the invention is to provide a novel pharmaceutical composition which can effectively increase the effective concentration of pharmaceutically active molecules in the brain.

In order to achieve the objects, the invention employs the following technical solutions:

Use of flavonol as a medicament for promoting the drug that has therapeutic or health effects on brain diseases to pass through the blood-brain barrier.

In particular, the flavonol is selected from kaempferide, myricetin, hesperidin, rutin, troxerutin, and hydroxy derivatives thereof. Further, the hydroxy derivatives of kaempferol, myricetin, hesperidin, rutin or troxerutin are their glycosides, esters and ethers.

The drug having a therapeutic or healthcare effect on the brain disease is selected from ginsenoside, stilbene glucoside of Radix polygoni multiflori, resveratrol, levodopa, edaravone, vinpocetine, nicergoline, citicoline, oxiracetam.

A composition, wherein its active ingredients are synergist flavonol and active molecules having a therapeutic or healthcare effect on brain diseases.

Preferably, the flavonol is selected from kaempferide, myricetin, hesperidin, rutin, troxerutin, and hydroxy derivatives thereof. Further, the hydroxy derivatives of kaempferol, myricetin, hesperidin, rutin or troxerutin are their glycosides, esters and ethers.

The drug having a therapeutic or healthcare effect on brain diseases is selected from ginsenoside, stilbene glucoside of Radix polygoni multiflori, resveratrol, levodopa, edaravone, vinpocetine, nicergoline, citicoline, oxiracetam.

Preferably, the active ingredients of the composition are:

kaempferide, total ginsenosides extract and stilbene glucoside at a mass ratio of 1:1˜5:1˜5 or
troxerutin, total ginsenosides extract and stilbene glucoside at a mass ratio of 1:1˜5:1˜2; or
at least one of rutin and troxerutin and vinpocetine, with the mass ratio of the sum of rutin and troxerutin to vinpocetine at 1:2˜5;
or myricetin and levodopa at a mass ratio of 1:2˜5;
or troxerutin and levodopa at a mass ratio of 1:2˜5;
or hesperidin and levodopa at a mass ratio of 1:1.5˜5;
or kaempferol and oxiracetam at a mass ratio of 1:2˜5;
or troxerutin and edaravone at a mass ratio of 1:2˜5;
or troxerutin and citicoline at a mass ratio of 1:2˜5.

The brain disease is selected from a cerebral ischemic injury disease or a neurodegenerative disease, such as ischemic stroke, stroke sequelae, vascular dementia, senile dementia, Parkinson's disease, etc.

The invention can achieve the following beneficial effects:

After long-term repeated studies, the inventors have found that some flavonol compounds, particularly kaempferide, rutin, troxerutin, myricetin, hesperidin, and hydroxy derivatives thereof, in particular their glycoside, ester, ether derivatives, can promote the drug molecules that have therapeutic or healthcare effect to enter brain tissues, to dramatically enhance their concentrations in the brain tissues and effective enhance the efficacy of drugs without increasing the plasma concentration.

The composition of the present invention can penetrate the BBB to achieve better therapeutic effect on brain diseases without causing unwanted side effects.

For the flavonol compounds, in particular kaempferide, rutin, troxerutin, myricetin, hesperidin, and hydroxy derivatives thereof, especially when their glycoside, ester, ether derivatives are used in combination with active ingredients such as ginsenoside, stilbene glucoside of Radix polygoni multiflori or resveratrol, levodopa, edaravone, vinpocetine, nicergoline, citicoline, oxiracetam, the active ingredients of these TCMs can penetrate the blood-brain barrier obviously, dramatically enhance the concentrations of ginsenoside, stilbene glucoside, or resveratrol, levodopa, edaravone, vinpocetine, nicergoline, citicoline, oxiracetam in brain tissues, promote neural stem cell proliferation and directional differentiation, anti-cerebral ischemic injury, improve learning and memory and resist senile dementia; in addition, their active ingredients are clear constituent and quality stability, which reduces the side effects of active ingredients in the peripheral tissues and plays a toxicity-reducing synergistic effect. With small doses, they can be made into a variety of controlled delivery formulations and used in large-scale production. It is a kind of excellent natural drug and/or healthcare food.

Meanwhile, the flavonol compounds used in the invention have a wide range of sources with low production cost. It is easy to be used widely.

### DETAILED DESCRIPTION OF THE EMBODIMENTS

The structural formula of flavonol is as follows:

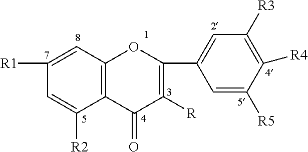

| Name of Compound | R | R1 | R2 | R3 | R4 | R5 |
|---|---|---|---|---|---|---|
| kaempfetol | —OH | —OH | —OH | —H | —OH | —H |
| Rutin | —O_glu-glu | —OH | —OH | —OH | —OH | —H |
| Troxerutin | —O-glu-glu | —O(CH₂)₂OH | —OH | —O(CH₂)₂OH | —(—OCH₂)₂OH | —H |
| Myricetin | —OH | —OH | —OH | —OH | —OH | —OH |
| Hesperidin | H | _manno-glu | OH | —OH | O— | —H |

Rutin and Kaempferol belong to flavonols, yellow needle crystal, with melting point of 276° C.-278° C. Kaempferol is slightly soluble in water, soluble in hot ethanol, ether and alkali. It has pharmacological effects such as anti-cancer, inhibiting fertility, antiepileptic, anti-inflammatory, antioxidant, antispasmodic, anti-ulcer, diuretics, and relieving cough, etc.

The pharmacological properties of Rutin (Rutin, phytomelin, vitamin P): melting point 176-8° C., [ ] 23D+13.82° C. (ethanol), [ ] 20D-39.43° C. (pyridine). 1 g of rutin is dissolved in 7 ml methanol, rutin is soluble in boiling water, but not soluble in water. Pharmacological effects: Rutin is a vitamin drug, and it has the effect of anti-inflammatory, anti-virus, reducing capillary permeability and brittleness, maintaining and restoring normal elasticity of capillaries. It can be used for prevention and treatment of hypertensive cerebral hemorrhage, diabetic retinal hemorrhage, hemorrhagic purpura and acute hemorrhagic nephritis, etc. In addition, it can be used as food antioxidants and pigments, has the effect of resisting skin aging and radiation, and scavenging free radicals.

Troxerutin (hydroxyethyl rutin, ethoxyethyl rutin) is a semi-synthetic flavonoid compound made from rutin by hydroxyethylation. It is often used as an anticoagulant and thrombolytic agent. This product can inhibit the erythrocyte and platelet agglutinations, prevent thrombosis, increase the oxygen content in the blood, improve microcirculation, promote neovascularization to enhance collateral circulation. It has a protective effect on endothelial cells, and it can resist against the vascular injury caused by serotonin and bradykinin, increase the capillary resistance, reduce capillary permeability, to prevent the edema caused by elevated vascular permeability and present a significant protective effect for acute ischemic brain injury. In addition, it can resist radiation injury, anti-inflammatory, anti-allergy, anti-ulcer, etc.

Myricetin is a flavonoids compound, yellow needle crystal (dilute ethanol), with melting point of 357˜360° C., slightly soluble in boiling water, soluble in ethanol, almost insoluble in chloroform and acetic acid. It has the following effects: 1. platelet activating factor (PAF) antagonistic effect; the cardiovascular pharmacological effects such as hypolipidemic (low-density lipoprotein cholesterol), anti-thrombosis, anti-myocardial ischemic and improving microcirculation, and promoting blood circulation to remove blood stasis; 2. Hypoglycemic effect: 3. anti-oxidation effect: Myricetin is inhibitors of cyclooxygenase 1 (COX-1), cyclooxygenase 2 (COX-2), 5-lipoxygenase (5-LOX). 4. Effect of protecting liver. 5. Anti-inflammatory and antibacterial effect. 6. Matrix metalloproteinase (MMP) inhibitors, the reagents for the prevention and treatment of cardiovascular diseases and tumors in the future. 7. The myricetin has a staining effect and it is not easy to fade.

Hesperidin (Hesperidin, dihydro flavonoid glycosides; naringin; citrusin) is a kind of flavonoid substance which is widely existing in citrus fruits. Its chemical structure has dihydroflavonoxone glycoside, and it has weak acidity. The crude product obtained by extraction is light yellow powder. Its pure product is white acicular crystal, slightly bitter taste, insoluble in water, almost insoluble in acetone, benzene, chloroform, slightly soluble in methanol, hot acetic acid, soluble in formamide, dimethylamide, and easily soluble in dilute alkali solution. The melting point of hesperidin with purity of 97% is within the range of 257˜260° C., and its molecular weight is 610.6. Hesperidin has the effects of maintaining osmotic pressure, increasing capillary toughness, reducing the capillary fragility, shortening the bleeding time and lowering cholesterol, and resisting oxidation; clinically, it is mainly used for adjuvant therapy of cardiovascular diseases such as hypertension, etc.

According to the existing documents and literatures, it has not been reported that kaempferide, rutin, troxerutin, myricetin, hesperidinand hydroxy derivatives thereof, especially their glycosides, esters and ether derivatives, can enhance the permeability of BBB and promote the entry of drug molecules into the brain tissues.

### Example 1

Flavonol kaempferide (content≧99.0%) 50, 100 g.

TCM active ingredient: ginsenoside Rb1 (referred to as Rb1, content≧95%) 200 g;

Weigh various extracts on the prescription and mix them uniformly according to the equal incremental method, to get the mixtures: LXN1-1 and LXN1-2.

### Pharmacodynamic Experiment:

Treatment Group: The LXN1-1 and LXN1-2 were made into suspension, administered to SD rats by intragastric administration. Five rats were given 20 ml/kg according to the dose of 125/150 mg/kg (equivalent to ginsenoside Rb1 100 mg/kg);

Control group: ginsenoside Rg1 extract was made into suspension, administered to SD rats by intragastric administration. Five rats were given ginsenoside Rb1 at the volume of 20 ml/kg according to the dose of 100 mg/kg;

The rats were drew blood 0.5 h after administration and then sacrificed under anaesthesia. Brain tissues were taken to prepare brain homogenates. The content of TCM active ingredient in the blood and brain tissues was detected by UPLC-MS.

In addition, 40 rats of MCAO forebrain ischemia were prepared, and they were administered solvent saline, 100 mg/kg LXN1 or ginsenoside Rb1 by gavage at 20 ml/kg, 10 rats per group; Animals were sacrificed 7 days after administration, to observe the improvement rate of cerebral infarction area.

The results were shown in Table 1.

TABLE 1
Results of determination of LXN1 by UPLC and BBB penetration

| | | | | | Rb1 content | Improvement rate |
|---|---|---|---|---|---|---|
| | Administration | Rb1 | | Rb1 plasma | in brain | of cerebral |
| | Dose | content | Kaempferide | concentration | tissues | infarction area |
| Drug | (mg/kg) | (%) | content (%) | (μg/ml) | (ng/g) | (%) |
| LXN1-2 | 150 | 68.12 | 33.0 | 3.52 ± 0.46 | 36.13 ± 3.29 | 71.2 ± 13.5 |
| LXN1-1 | 125 | 79.8 | 20.0 | 3.41 ± 0.46 | 16.13 ± 3.29 | 52.1 ± 9.9 |
| Rb1 | 100 | 100 | 0 | 3.35 ± 0.68 | 4.12 ± 1.13 | 22.1 ± 9.1 |
| Saline | 0 | 0 | 0 | 0 | 0 | 0 |

The results showed that, when LXN1 and ginsenoside Rb1 with equal dose of Rb1 were administered, there was no significant difference of plasma concentration between them, but the ginsenoside Rb1 content of LXN1 in brain tissues was 5/9 time higher than that of rats administered ginsenoside Rb1 alone, suggesting that the combination of flavonol kaempferide and ginsenoside Rb1 could promote the active ingredient Rb1 to pass through the blood-brain barrier.

### Example 2

Rutin (content≧99.0%) 80, 100 g

TCM active ingredient: ginsenoside Rg1 (referred to as Rg1, content≧95%) 200 g;

Weighing various extracts on the prescription and mixing them uniformly according to the equal incremental method, and get the mixtures: LXN11-1 and LXN11-2.

### Pharmacodynamic Experiment:

Treatment Group: The LXN11-1 and LXN11-2 were made into suspension, administered to SD rats. Five rats were given 20 ml/kg with the dose of 140/150 mg/kg (equivalent to ginsenoside Rg1 100 mg/kg) in each group;

Control group: ginsenoside Rg1 extract was made into suspension. 5 SD rats were administered the suspension at the dose of 100 mg/kg and the volume of 20 ml/kg by intragastric administration;

The rats were sacrificed 0.5 h after administration by drawing blood under anaesthesia. Brain tissues were taken to prepare brain homogenates. The content of TCM active ingredient in the blood and brain tissues was detected by UPLC-MS.

In addition, 40 rats of MCAO forebrain ischemia were prepared, and they were administered with solvent saline, 100 mg/kg LXN11 and ginsenoside Rg1 by gavage at 20 ml/kg, 10 rats per group; Animals were sacrificed 7 days after administration, to observe the improvement rate of cerebral infarction area.

The results were shown in Table 2.

TABLE 2
Determination of LXN11 by UPLC and BBB penetration

| | | | | | Rb1 content | Improvement rate |
|---|---|---|---|---|---|---|
| | | Rb1 | Rutin | Rb1 plasma | in brain | of cerebral |
| | Administration | content | content | concentration | tissues | infarction area |
| Drug | Dose (mg/kg) | (%) | (%) | (μg/ml) | (ng/g) | (%) |
| LXN11-2 | 150 | 66.12 | 33.0 | 5.52 ± 0.56 | 69.6 ± 7.4 | 73.2 ± 11.5 |
| LXN11-1 | 140 | 71.3 | 28.57 | 5.41 ± 0.49 | 66.3 ± 7.9 | 62.8 ± 9.3 |
| Rg1 | 100 | 100 | 0 | 4.85 ± 0.63 | 7.2 ± 1.1 | 26.1 ± 7.1 |
| Saline | 0 | 0 | 0 | 0 | 0 | 0 |

The results showed that, when LXN11 and ginsenoside Rg1 with equal dose of Rg1 were administered, there was no significant difference of plasma concentration between them, but the ginsenoside Rg1 content of LXN11 in brain tissues was 9 times higher than that of rats administered ginsenoside Rg1 alone, suggesting that the combination of rutin and ginsenoside Rg1 could promote the active ingredient Rg1 to pass through the blood-brain barrier and enhance the effect of protecting cerebral ischemia.

### Example 3

Troxerutin (content≧95.0%) 50 or 100 g;

TCM active ingredient: extract of Radix polygoni multiflori (stilbene glucoside, EB, content≧50%) 200 g.

Weigh various extracts on the prescription and mix them uniformly according to the equal incremental method, to get the mixtures: LXN2-1 and LXN2-2.

### Pharmacodynamic Experiment:

Treatment Group: LXN2-1 and LXN2-2 were made into suspension. 5 SD rats were administered the suspension with the dose of 100 mg/kg stilbene glucoside and the volume of 20 ml/kg by intragastric administration;

Control group: stilbene glucoside extract was made into suspension. 5 SD rats were administered the suspension at the dose of 100 mg/kg stilbene glucoside and the volume of 20 ml/kg by intragastric administration;

The rats were drew blood 0.5 h after administration and then sacrificed under anaesthesia. Brain tissues were taken to prepare brain homogenates. The content of stilbene glucoside in the blood and brain tissues were detected by UPLC-MS.

In addition, 40 rats of 4-VO local cerebral ischemia were prepared and they were administered with solvent saline, 100 mg/kg LXN2-1, LXN2-1 and stilbene glucoside by gavage at the volume of 20 ml/kg, 10 rats per group; Animals were sacrificed 7 days after administration, to observe the improve rate of nerve regeneration, learning and memory.

The results were shown in Table 3.

TABLE 3
Determination of LXN2 by UPLC and BBB penetration

| | | | | Plasma | Content of | | |
|---|---|---|---|---|---|---|---|
| | Content of | | Dose of | concentration | stilbene | | Improvement |
| | stilbene | Content of | stilbene | of stilbene | glucoside in | Nerve | rate of |
| | glucoside | troxerutin | glucoside | glucoside | brain tissues | regeneration | learning and |
| Drug | (%) | (%) | mg/kg | (μg/ml) | (ng/g) | rate (%) | memory (%) |
| LXN2-1 | 30.5 | 33.0 | 100 | 3.52 ± 0.46 | 360.3 ± 32.3 | 280.1 ± 56.3 | 85.6 ± 24.3 |
| LXN2-2 | 30.5 | 20.0 | 100 | 3.52 ± 0.46 | 183.3 ± 31.8 | 201.6 ± 43.4 | 53.8 ± 19.2 |
| Stilbene | 100 | 0 | 100 | 3.35 ± 0.68 | 40.2 ± 16.1 | 81.2 ± 35.6 | 31.6 ± 21.0 |
| glucoside | | | | | | | |

The results showed that, when LXN2-1/2 and stilbene glucoside extract with equal dose of stilbene glucoside wer administered, there was no significant difference of plasma concentration between them, but the content of stilbene glucoside of LXN2-1/2 in brain tissues was 4.5/9 times higher than that of rats administered stilbene glucoside alone, suggesting that the combination of flavonol derivative troxerutin and stilbene glucoside could promote the active ingredient stilbene glucoside to pass through the blood-brain barrier, show the brain-targeting effect, and enhance the efficacy of stilbene glucoside in suppressing cerebral ischemia injury and improve the learning and memorization ability.

### Example 4

Resveratrol (content of 96%) 100 g;

Rutin (content≧96.0%) 50 g.

Weigh two kinds of extracts by the prescription and mix them uniformly according to the equal incremental method, to get the mixture LXN3.

### Pharmacodynamic Experiment:

Treatment Group: LXN3 was made into suspension. 5 SD rats were administered the suspension at the dose of 100 mg/kg resveratrol and the volume of 20 ml/kg by intragastric administration;

Control group: Resveratrol was made into suspension. 5 SD rats were administered the suspension at the dose of 100 mg/kg resveratrol and the volume of 20 ml/kg by intragastric administration;

The rats were derw blood 0.5 h after administration and then sacrificed under anaesthesia. Brain tissues were taken to prepare brain homogenates. The content of resveratrol in the blood and brain tissues were detected by UPLC-MS.

In addition, 30 rats of MCAO forebrain ischemia were prepared, and they were administered solvent saline, 100 mg/kg LXN3, and resveratrol by gavage at 20 ml/kg, 10 rats per group; Animals were sacrificed 7 days after administration, to observe the improvement rate of cerebral infarction area.

The results were shown in Table 4.

TABLE 4
Determination of LXN3 by UPLC and BBB penetration

| | | | | | Improvement rate |
|---|---|---|---|---|---|
| | Resveratrol | Rutin | Plasma | Resveratrol content | of cerebral |
| | content | content | concentration of | in brain tissues | infarction area |
| Drug | (%) | (%) | resveratrol (μg/ml) | (ng/g) | (%) |
| LXN3 | 68.12 | 31.0 | 5.66 ± 1.21 | 26.34 ± 3.29 | 62.8 ± 11.3 |
| Resveratrol | 100 | 0 | 5.58 ± 1.28 | 3.12 ± 1.29 | 20.1 ± 8.6 |

According to the data in Table 4, when LXN3 and resveratrol with equal dose of resveratrol were administered, there was no significant difference in plasma concentration between them, but the resveratrol content of LXN3 in brain tissues was 8 times higher than that of rats administered resveratrol alone, and the improvement rate of cerebral infarction area in rats of MCAO cerebral ischemia was increased by more than 3 times, suggesting that the combination of flavonol compound rutin and resveratrol could promote the active ingredient to pass through the blood-brain barrier with the brain-targeting effect and enhance the efficacy against cerebral ischemic injury.

### Example 5

LXN4: commercially available kaempferide extract (content≧60%), total ginsenosides extract (content≧60%), stilbene glucoside of Radix polygoni multiflori (content≧50%), composed at the mass ratio of 1:2:1.

LXN5: Commercially available troxerutin (content≧95%), total ginsenosides extract (content≧90%), stilbene glucoside (content≧60%), composed at the mass ratio of 1:1.75:0.75.

RSSW: total ginsenosides extract (content≧90%), stilbene glucoside (content≧90%), composed at the mass ratio of 1:1.

### Pharmacodynamic Experiment:

Treatment Group: LXN4, LXN5, RSSW were made into suspension. Five SD rats were administered the suspension at the dose of 100 mg/kg and the volume of 20 ml/kg by intragastric administration;

Control group: The total ginsenosides extract was made into suspension. Five SD rats were administered the suspension at the dose of 50 mg/kg and the volume of 20 ml/kg by intragastric administration;

The rats were derw blood 0.5 h after administration and then sacrificed under anaesthesia. Brain tissues were taken to prepare brain homogenates. The content of active ingredient in the blood and brain tissues were detected by UPLC-MS.

In addition, 40 rats of MCAO forebrain ischemia were prepared, and they were administered solvent saline, 100 mg/kg LXN4, LXN5, RSSW, total ginsenosides by gavage at 20 ml/kg, 10 rats per group; Animals were sacrificed 7 days after administration, to observe the improvement rate of cerebral infarction area and nerve regeneration, learning and memorization ability.

The results were shown in Tables 5-7:

TABLE 5
The active ingredient content (μg/ml) in the blood 0.5 h
after administered different compositions

| Test Item | Total ginsenosides | RSSW | LXN4 | LXN5 |
|---|---|---|---|---|
| Stilbene glucoside | | 48.5 | 16.8 | 13.1 |
| Ginsenoside Rg1 | 15.3 | 15.1 | 15.8 | 15.1 |
| Ginsenoside Rd | 4.6 | 4.5 | 4.1. | 4.0 |
| Ginsenoside Rb1 | 22.1 | 22.0 | 21 | 21.5 |
| Ginsenoside Re | 4.1 | 4.4 | 4.8 | 4.1 |
| Kaempferol | | | 20.1 | 0.1 |

TABLE 6
The active ingredient content (ng/ml) in the brain tissue 0.5 h
after administered different compositions

| Test Item | Total ginsenosides | RSSW | LXN4 | LXN5 |
|---|---|---|---|---|
| Stilbene glucoside | | 4.8 | 36.8 | 132.1 |
| Ginsenoside Rg1 | 2.20 | 15.1 | 95.8 | 115.6 |
| Ginsenoside Rd | 0.46 | 4.5 | 24.1. | 29.5 |
| Ginsenoside Rb1 | 2.21 | 22.0 | 121 | 128 |
| Ginsenoside Re | 0.81 | 1.05 | 9.8 | 8.9 |
| Kaempferol | | | 12 | 0.2 |

TABLE 7
Protective effect of different compositions on cerebral ischemia
7 days after administration in rats

| | Total | | | |
|---|---|---|---|---|
| Test Item | ginsenosides | RSSW | LXN4 | LXN5 |
| Infarction area (%) | 43.14 | 39.42 | 24.1 | 21.21 |
| Nerve regeneration rate (%) | 87 | 91 | 192.20 | 322.60 |
| Improvement rate of learning | 19.96 | 24.46 | 78.46 | 72.46 |
| and memory | | | | |
| Space exploration distance | 26.21 | 44.21 | 76.21 | 81.12 |
| (cm) | | | | |

According to the data in tables 5-7, when administered different compositions with equal dose of total ginsenosides and stilbene glucoside, the contents of total ginsenosides and stilbene glucoside in the blood showed no significant difference, but when combined with flavonols, the contents of ginsenosides and stilbene glucosides increased dramatically by 5-40 times in brain tissue.

The pharmacological evaluation showed that, 7 days after LXN4 and LXN5 administered, the infarct area was reduced, the cerebral nerve regeneration rate was increased, the learning and memory improvement rate was increased and the space exploration distance (cm) was extended, and the drug efficacy was stronger than the total ginsenosides or RSSW alone. Flavonols compounds could promote the TCM active ingredient to pass through the blood-brain barrier, enhance the anti-cerebral ischemic injury and nerve protection, and improve learning and memory function.

### Application in the Clinical Treatment of Sequelae of Ischemic Stroke

### 1. Clinical Data and Method

### 1.1 General Information

Ninety patients with sequelae of cerebral infarction enrolled in our hospital from February 2010 to October 2014 were selected as the subjects, and randomly divided into total ginsenosides capsule group, RSSW capsule group and LXN5 capsule group, 30 cases in each group. In the total ginsenosides capsule group, there were 30 cases, including 16 males and 14 females; aged 38-73 (51.8±6.7) years, and duration of 37- to 84 (51.6±7.4) d. In the RSSW capsule group, there were 30 cases, including 16 males and 14 females; age 37-74 (52.1±7.2) years; duration of 37˜85 (52.1±9.3) d. In the LXN5 capsule control group, there were 30 cases, including 16 males and 14 females; aged 37˜75 (52.1±8.1) years, duration 39˜84 (52.1±8.9)d; there were no significant difference in the age, gender, duration of disease, body mass, history of health, history of medication, history of illness, type of stroke and other demographic data of patients between these three groups (P>0.05) and they were comparable.

### 1.2 Inclusion Criteria

(1) The diagnosis of western medicines comply with the criteria in the Diagnosis of various cerebrovascular diseases amended in 1995 Fourth National Cerebrovascular Disease Conference of Chinese Medical Association according to [1], and diagnosed by skull CT or MRI; (2) remission after symptomatic treatment in the acute phase, without other severe complications; (2) TCM diagnosis comply with the Criteria for the evaluation of efficacy of stroke diagnosis drafted by Research Group on Acute Encephalopathy of State Administration of Traditional Chinese Medicine according to [2]; (3) aged 40-80 years; (4) The stage of disease: recovery stage (2 weeks-6 months); (5) sequelae such as hemiplegia, limb numbness, facial paralysis, unsmooth speech, etc.; (5) patients were willing to sign the ICF.

### 1.3 Exclusion Criteria

(1) Patients with acute stroke patients; transient ischemic attack, cerebral hemorrhage or asymptomatic cerebral infarction; (2) severe joint deformity affecting the recovery of functions; (3) Patients with insufficiency of heart, liver and kidney, etc.; (3) Patients who were not willing to cooperate; (4) pregnant or breast-feeding women, with allergic constitution or allergic to the known drug compositions; (5) Patients with unstable vital signs that need emergency care; (6) Patients with mental disorder or disturbance of consciousness; (7) Patients with secondary stroke caused by tumors and blood diseases, etc.;

### 1.4 Treatment

The three groups of patients were administered total ginsenosides capsules, RSSW capsules and LXN5 capsules after they were included in the groups, for 14 consecutive days as a course of treatment, four courses in total.

### 1.5 Observation Indicators

Observation indicators: The neurological severity scores [4] and scores of daily activities [5] of patients before and after treatment were observed and recorded.

### Response Evaluation Criteria:

Cured The clinical symptoms and signs of TCM syndrome disappeared or basically disappeared after treatment, with the therapeutic index of 95%; markedly effective: signs were significantly improved after treatment, 70%≦the therapeutic index<95%; effective: the signs were improved after treatment, 30%≦the therapeutic index<70%; Ineffective: no signs were improved after treatment, the therapeutic index<30%.

Overall response rate=number of (cured+markedly effective+effective)cases/total number of cases

### 1.6 Statistical Method

Data processing was performed by SPSS 19.0 software package. χ2 test was adopted for comparison of count data, and the measurement data were expressed by (x̅±s). t test was performed for comparison between groups. P<0.05 was considered statistically significant difference.

### Efficacy Results

### 2.1 Neurological Severity Scores (NIHSS)

The overall response rates of patients in the three groups were improved, and the neurological functions were improved significantly after treatment. The comparison of efficacy among the three groups: total ginsenosides capsules<RSSW capsules<LXN5 capsules, without significant difference among them, as shown in Table 8.

TABLE 8
Comparison of neurological severity scores (NIHSS)
of patients in the three groups

| | Total | | | |
|---|---|---|---|---|
| | number | Before | | |
| Group | of cases | treatment | After treatment | P value |
| LXN5 capsule | 30 | 17.2 ± 5.7 | 7.5 ± 4.2** | <0.05 (0.011) |
| RSSW capsule | 30 | 17.1 ± 5.8 | 11.1 ± 4.2* | <0.05 (0.028) |
| Total | 30 | 16.9 ± 6.1 | 13.1 ± 2.3* | <0.05 (0.045) |
| ginsenosides | | | | |
| capsule | | | | |
| p | | >0.05 | <0.05 | |

### 2.3 Assessment of Daily Living Capacity (BL)

The daily living capacity of patients in the two groups was significantly improved after treatment. The efficacy of brain-targeting therapy was superior to the control group, with statistically significant difference, as shown in Table 9.

TABLE 9
Daily living ability status (BL) scores of patients in three groups

| | Total | | | |
|---|---|---|---|---|
| | number | Before | | |
| Group | of cases | treatment | After treatment | P value |
| LXN5 capsule | 30 | 53.2 ± 19.7 | 91.5 ± 15.4** | <0.05 (0.009) |
| RSSW capsule | 30 | 53.1 ± 21.0 | 85.6 ± 14.2* | <0.05 (0.019) |
| Total | 30 | 52.0 ± 22.8 | 78.1 ± 12.3* | <0.05 (0.045) |
| ginsenosides | | | | |
| capsule | | | | |
| p | | >0.05 | <0.05 | |

Compared with itself before treatment: *p < 0.05.

### 2.4 Response for Stroke

The overall response rates in the 3 groups were effective; after treatment, the response on stroke was significant improved. The response rate of LXN5 capsule was 90%, superior to RSSW capsule and total ginsenosides capsule, with statistically significant difference (chi-square test, * p<0.05), as shown in Table 10 for details.

TABLE 10
Response for Stroke

| | Total | | | | | |
|---|---|---|---|---|---|---|
| | number | | Markedly | | | ORR/ |
| Group | of cases | Cured | effective | Effective | Ineffective | % |
| LXN5 | 30 | 8 | 10 | 9 | 3 | 90.00* |
| capsule | | | | | | |
| RSSW | 30 | 4 | 10 | 10 | 6 | 80.00 |
| capsule | | | | | | |
| Total | 30 | 2 | 11 | 9 | 7 | 73.33 |
| ginsenosides | | | | | | |
| capsule | | | | | | |

The above data showed that the brain-targeting compatible LXN5 had significantly improved the treatment effect of stroke and stroke sequelae.

### Treatment of Parkinson's Disease

(1) Clinical Data and Methods

Diagnostic criteria: Patients with primary PD diagnosed according to the Guidelines for Treatment and Diagnosis of Parkinson's Disease in 2010 (Beijing Union Medical College Hospital. Guidelines for Treatment and Diagnosis of Parkinson's Disease [J]. Chinese Journal for Clinicians, 2010, 38 (2): 77-79.)

Exclusion criteria: (a) Parkinson's disease due to severe heart, lung and renal dysfunction, secondary to cerebrovascular disease, trauma and other neurological and psychiatric disorders; (b) Parkinson-plus-syndromes; (c) patients with malignancy, disability and other neurological, blood, endocrine primary diseases; (d) patients with a history of symptomatic Parkinson's disease, psychosis, drug abuse and alcohol abuse.

The main symptoms of traditional Chinese medicine: reference to Guidelines for Clinical Trial of New TCM, Guidelines for Clinical Trials of New TCM in Treatment of Senile Dementia.

(2) Sources of Patients: Outpatients and inpatients from First Affiliated Hospital of Guangdong College of Pharmacy.

Profile of patients: All selected patients were diagnosed with Parkinson's disease according to the Parkinson's disease diagnostic criteria in clinical examination during 2010-2014. Patients were from department of neurology, a total of 123 cases, including 66 males, 57 females; aged 51-76 years, and 63.1 years on average, course of disease 1-3 years, shortest duration of 1 year, and longest duration of 15 years, 3.8 years on average.

All eligible cases were randomly divided into treatment group, control group 1 and control group 2. of which:

Total ginsenoside capsule group: 41 cases, including 23 males and 18 females; duration of 1-12 years, 3.7 years on average; aged 51-75 years, 60.4 years on average.

RSSW capsule group: 41 cases, including 22 males and 19 females; duration of 1-12 years, 3.7 years on average; aged 51-76 years, 60.2 years on average.

LXN5 capsule group: 41 cases, including 22 males and 19 females; duration of 1-12 years, 3.8 years on average; aged 51-75 years, 59.5 years on average.

By statistical analysis of the above 3 groups of patients, the differences in the gender, duration and age were not statistically significant (P>0.05) and were comparable. All subjects were informed and signed the informed consent.

(3) Treatment Regimens

Total ginsenoside capsule group: Patients took capsules made in Example 4, 250 mg/capsule, 2 capsules per time, 3 times a day.

RSSW capsule group: Patients took capsules made from following method, 250 mg/capsule, 2 capsules per time, 3 times a day.

LXN5 capsule group: orally taken (specification: 250 mg/tablet), 125˜250 mg, 3 times/day, individualized administration according to the condition of disease.

Fifteen days as a course in all groups, a total of 9 months for medication; and during the period, other drugs regulating cerebral vasodilation, brain cell metabolism and neurological functions were discontinued.

In all groups, their curative effects were evaluated after treatment for 2 weeks, 1 month, 3 months, 6 months and 9 months.

(4) Observation Indexes

\textcircled{1} Safety observation: including general life signs (blood pressure, heart rate, respiration), blood routine, urine routine, heart and liver function tests and adverse reactions (excitement, irritability, dry mouth, tongue dryness). Twice per day for general life signs observation. Outpatients must be told the detection method and their families must be told to take records; for other indexes, examined once before and after treatment respectively.
\textcircled{2} Curative effect: Main symptoms before and after treatment (finger and limb vibration, shaking, muscle stiffness that caused non-autonomic activities of a part of limbs or all limbs), signs and laboratory indicators (hemorheology, cerebral blood flow, Platelet adhesion and aggregation rate), etc.

The curative effect was assessed according to the unified Parkinson's Disease Rating Scale (UPDRS).

Efficacy index={(pre-treatment score−post-treatment score)/pre-treatment score×100%.

Cured when efficacy index≧85%; markedly effective when efficacy index was within 70-84%; effective when efficacy index was within 20%-69%; ineffective when efficacy index<20%.

\textcircled{1} Cured clinically: symptoms such as non-autonomic activities of a part of limbs or all limbs caused by finger and limb vibration, shaking, muscle stiffness disappeared, able to walk independently, take care of themselves, the upper and lower limbs muscle strength recovered to grades 4-5.
\textcircled{2} markedly effective: Symptoms such as non-autonomic activities of a part of limbs or all limbs caused by finger and limb vibration, shaking, muscle stiffness were improved significantly, able to walk on foot, and the upper and lower limbs muscle strength recovered to above grade 2.
\textcircled{3} effective: Symptoms such as non-autonomic activities of a part of limbs or all limbs caused by finger and limb vibration, shaking, muscle stiffness were improved; the upper and lower limbs muscle strength recovered to above grade 1.
\textcircled{4} Ineffective: Symptoms such as non-autonomic activities of a part of limbs or all limbs caused by finger and limb vibration, shaking, muscle stiffness were not improved after treatment.

The severity of motor symptoms was assessed by the Unified Parkinson's Disease Rating Scale (UPDRS) Part III and the Hoehn-Yahr Rating Scale, and the patients' daily living ability was assessed by the Unified Parkinson's Disease Rating Scale (UPDRS) Part II and ADCS-ADL. The patients' mental, behavioral, and emotional states were assessed by Unified Parkinson's Disease Rating Scale (UPDRS) Part I.

The changes of main TCM symptoms and signs should be evaluated comprehensively and the changes in motor function should be emphasized.

(5) Statistical Method: T Test was Adopted for Measurement Data, and Chi-Square Test was Adopted for Count Data; the Ridit Test was Adopted for Ranked Data.

(6) Analysis of Curative Effect

Table 11 showed that the drug in the invention could effectively improve the main symptoms and signs of Parkinson's disease. According to the recognized efficacy standard, the overall treatment of Parkinson's disease had achieve good clinical efficacy; and according to the comparison between groups, the efficacy of the LXN5 capsule was superior to the RSSW capsule, while that of the RSSW capsule was superior to the total ginsenoside capsule, with statistically significant difference (p<0.01).

TABLE 11
Curative effect for PD in the three groups

| | | | | | | | | | | Overall |
|---|---|---|---|---|---|---|---|---|---|---|
| | Number | | | Markedly | | | | | | response |
| | of cases | Clinically cured | | effective | | Effective | | Ineffective | | rate |
| Group | (n) | n | % | n | % | n | % | n | % | % |
| LXN5 capsule | 41 | 3 | 7.8 | 20 | 48.5 | 15 | 39.0 | 3 | 10.0 | 92.68 |
| RSSW capsule | 41 | 1 | 2.44 | 18 | 43.9 | 14 | 34.1 | 7 | 17.1 | 82.9 |
| Total | 41 | 0 | 0 | 19 | 46.3 | 13 | 31.7 | 9 | 22.0 | 78.05 |
| ginsenoside | | | | | | | | | | |
| capsule | | | | | | | | | | |

Compared with control group 2, p < 0.05.

The clinically cured, markedly effective and effective cases were used as the basis for calculating the overall response rate. For comparison of clinical efficacy in the three groups, the overall response rate (ORR) of the treatment group and control group 1 was 92.68% and 82.9% respectively, and the ORR of control group 2 was 78.05%. The comparison between two groups showed significant difference (p<0.05).

A. Psychiatric, behavioral, and emotional scores (UPDRS-1): After treatment, the psychiatric, behavioral, and emotional scores of the WSXN group improved significantly. The efficacy of the LXN5 capsule was superior to the RSSW capsule, while that of the RSSW capsule was superior to the total ginsenoside capsule, showing statistically significant difference (p<0.05), as shown in Table 12.

B. Daily Living Activity (UPDRS-II) Score: After treatment, the efficacy of the LXN5 capsule was superior to the RSSW capsule, while that of the RSSW capsule was superior to the total ginsenoside capsules, showing statistically significant difference (p<0.05), as shown in Table 13.

C. Motor function examination (UPDRS-III) score: After treatment, the scores of various motor function examinations were significantly improved, and the efficacy of the LXN5 capsule was superior to the RSSW capsule, while that of the RSSW capsule was superior to the total ginsenoside capsules, showing statistically significant difference (p<0.05), as shown in Table 14.

D. Motor complications (UPDRS-IV) scores: After treatment, the motor complications scores in various groups were significantly improved compared with the control group 2. The efficacy of the LXN5 capsule was superior to the RSSW capsule, while that of the RSSW capsule was superior to the total ginsenoside capsule, showing statistically significant difference (p<0.05), as shown in Table 15.

E. Scores of Quality of Life Scale (PDQ): After treatment, the scores of quality of life scale in various groups were significantly improved compared with the control group 2. The efficacy of LXN5 capsules was superior to RSSW capsules while that of the RSSW capsule was superior to the total ginsenoside capsule, showing statistically significant difference (p<0.05), as shown in Table 16.

The changes of scale scores before and treatment were shown in Tables 12-16.

TABLE 12
Changes of psychiatric, behavioral, and emotional scores (UPDRS-1)
before and after treatment (x̅ ± s)

| | | | | 1 month | | 3 months | | 6 months | | 12 months | |
|---|---|---|---|---|---|---|---|---|---|---|---|
| | | Before | | after | | after | | after | | after | |
| Group | n | treatment | | treatment | | treatment | | treatment | | treatment | |
| LXN5 capsule | 41 | 3.66 ± | 1.02 | 2.77 ± | 0.64 | 1.51 ± | 0.52 | 1.18 ± | 0.52 | 1.03 ± | 0.51** |
| RSSW capsule | 41 | 3.63 ± | 0.97 | 3.03 ± | 0.71 | 1.96 ± | 0.54 | 1.67 ± | 0.53 | 1.31 ± | 0.53** |
| Total ginsenoside | 41 | 3.59 ± | 1.01 | 3.25 ± | 0.81 | 2.39 ± | 0.71 | 1.86 ± | 0.53 | 1.74 ± | 0.54 |
| capsule | | | | | | | | | | | |

Note:
* compared with total ginsenoside capsule, p < 0.05, **p < 0.01.

TABLE 13
Changes of Daily Living Activity (UPDRS-II) Score before and after treatment
(x̅ ± s)

| | | | 1 month | 3 months | 6 months | 12 months | |
|---|---|---|---|---|---|---|---|
| | | Before | after | after | after | after | |
| Group | n | treatment | treatment | treatment | treatment | treatment | P |
| LXN5 | 41 | 15.63 ± 5.56 | 13.89 ± 5.01 | 11.14 ± 5.11 | 8.83 ± 4.16* | 6.16 ± 3.61** | <0.05 |
| capsule | | | | | | | |
| RSSW | 41 | 15.61 ± 5.68 | 13.96 ± 5.13 | 11.96 ± 4.83 | 10.03 ± 4.11* | 8.29 ± 3.61* | <0.05 |
| capsule | | | | | | | |
| Total | 41 | 15.64 ± 5.67 | 14.32 ± 5.21 | 12.48 ± 5.02 | 11.18 ± 3.31 | 10.36 ± 3.43 | <0.05 |
| ginsenoside | | | | | | | |
| capsule | | | | | | | |

TABLE 14
Changes of motor function examination (UPDRS-III) scores before and after
treatment (x̅ ± s)

| | | | 1 month | 3 months | 6 months | 12 months |
|---|---|---|---|---|---|---|
| | | Before | after | after | after | after |
| Group | n | treatment | treatment | treatment | treatment | treatment |
| LXN5 capsule | 41 | 38.61 ± 9.21 | 31.96 ± 8.58 | 20.62 ± 6.54* | 16.63 ± 6.13* | 13.16 ± 7.03* |
| RSSW capsule | 41 | 38.16 ± 9.09 | 32.56 ± 8.63 | 25.31 ± 6.58* | 22.15 ± 6.54 | 19.58 ± 7.23 |
| Total ginsenoside | 41 | 37.93 ± 10.28 | 34.15 ± 7.92 | 28.93 ± 6.56 | 24.81 ± 6.52 | 21.13 ± 6.76 |
| capsule | | | | | | |

TABLE 15
Changes of motor complications (UPDRS-IV) scores before and after treatment
(x̅ ± s)

| | | | 1 month | 3 months | 6 months | 12 months |
|---|---|---|---|---|---|---|
| | | Before | after | after | after | after |
| Group | n | treatment | treatment | treatment | treatment | treatment |
| LXN5 capsule | 41 | 3.06 ± 1.01 | 2.28 ± 0.83 | 1.67 ± 0.64* | 1.28 ± 0.65** | 1.03 ± 0.53* |
| RSSW capsule | 41 | 3.06 ± 1.01 | 2.56 ± 0.82 | 2.08 ± 0.71 | 1.79 ± 0.71* | 1.41 ± 0.58* |
| Total ginsenoside | 41 | 3.59 ± 1.02 | 2.91 ± 0.86 | 2.46 ± 0.61 | 2.18 ± 0.56 | 1.77 ± 0.53 |
| capsule | | | | | | |

TABLE 16
Changes of daily life satisfaction (LSIB) scores before and after treatment (x̅ ± s)

| | | | 1 month | 3 months | 6 months | 12 months |
|---|---|---|---|---|---|---|
| | | Before | after | after | after | after |
| Group | n | treatment | treatment | treatment | treatment | treatment |
| LXN5 capsule | 41 | 12.56 ± 2.01 | 14.41 ± 3.73 | 16.83 ± 3.56 | 18.71 ± 3.95 | 20.68 ± 4.63 |
| RSSW capsule | 41 | 12.49 ± 2.11 | 13.78 ± 3.58 | 15.64 ± 3.61 | 17.51 ± 3.79 | 18.25 ± 4.31 |
| Total | 41 | 12.59 ± 2.12 | 13.05 ± 3.74 | 14.91 ± 3.56 | 16.85 ± 3.65 | 17.04 ± 3.54 |
| ginsenoside | | | | | | |
| capsule | | | | | | |

The drugs in the invention have a markedly ameliorating effect on dyskinesias of Parkinson's disease (PD). As shown from tables 11-16, after 121 patients were treated with LXN5 capsules, RSSW capsules, total ginsenoside capsules for 24 courses, the treatment scale scores of the three groups of drugs were significantly increased (p<0.01), indicating that the drugs had the effect of relieving muscle tremor and muscle stiffness, and dramatically improving the symptoms such as unsmooth speech, insomnia, irritability, dysphoria, tongue stasis. The effective rates of LXN5 capsules, RSSW capsules and total ginsenoside capsules on treatment of 41 patients with Parkinson's disease (PD) were 90%, 80% and 73.3% respectively, although the cure rates were 7.8% and 2.5% and the markedly effective rates were 48.5% and 46.3%. The efficacy of the brain-targeting drug in the treatment of PD was higher than the overall response rate of conventional medobarb tablet (70.05%), showing significant difference (p<0.05).

### Effects of LXN5 Capsules, RSSW Capsules and Total Ginsenoside Capsules on Vascular Dementia

(1) Clinical Data and Methods

Conditions of Cases: All 90 cases were the inpatients of the Department of Neurology, including 51 males and 39 females; aged 58-76 years, 63.9 years on average; the shortest duration of 1 year, longest duration of 10 years, 3.5 years on average. After clinical examination and neurological scale test, these patients were diagnosed by skull CT or MR.

Diagnostic criteria: The diagnostic criteria for vascular dementia in DSM-IV.

Diagnostic criteria: The diagnostic criteria for vascular dementia in DSM-IV.

Exclusion criteria: Patients with serious neurological, blood, endocrine and other primary diseases, and patients of senile dementia (the score of Hijinski Ischemic Index (HIS)<7, total scores of 18).

Selection of scales: 1. American Simple Intelligence Scale, a total of 30 points, if the score<16 points, it is disturbance of intelligence; 2. Japan Hasegawa Dementia Scale, a total score of 30 points, if the score<16 points, it is dementia; 3. Hijinski Ischemic Index (HIS), a total score of 18 points, if the score>7 points, it is vascular dementia, and if the score<7 points, it is senile dementia.

The following symptoms were used as the observation indexes according to the clinical experiences: sluggish, unsmooth speech, or few words or language inversion, forgetfulness, insomnia, dizziness, headache, tongue stasis.

Efficacy criteria: Comprehensive assessment method was adopted. The changes in the intelligent state and signs before and after treatment were comprehensively assessed and the change of intelligence was the focus. For cured patients, the score of Hasegawa Dementia Scale was increased to the normal value, for markedly effective, the score was increased by more than 5 points, for effective, the score was increased by less than 5 points, and for ineffective, the score was decreased rather than increased.

Medication: LNX5 capsules were taken in the morning, noon and evening every day, 2 capsules each time. For Renshen Shouwu capsules and total ginsenoside capsules, 0.3 g per capsule, taken orally, 3 times a day, taken before meals. One course lasted 2 months, 3 courses in total. During the period, other drugs regulating cerebral vasodilation, brain cell metabolism and neurological functions were discontinued.

(2) Efficacy and Results

The scale scores before and after treatment: (see Tables 17-19).

TABLE 17
Simple intelligent scale scores before and after treatment (x̅ ± s)

| Drug | n | Before treatment | After treatment | P |
|---|---|---|---|---|
| LXN5 capsule | 30 | 13.66 ± 5.11 | 19.63 ± 5.03 | <0.05 |
| RSSW capsule | 30 | 13.53 ± 5.02 | 18.35 ± 4.86 | <0.05 |
| Total ginsenoside capsule | 30 | 13.58 ± 5.03 | 17.98 ± 4.06 | <0.05 |

TABLE 18
Hasegawa Dementia Scale Scores before and after treatment (x̅ ± s)

| Drug | n | Before treatment | After treatment | P |
|---|---|---|---|---|
| LXN5 capsule | 30 | 12.85 ± 4.66 | 18.86 ± 6.17 | <0.05 |
| RSSW capsule | 30 | 12.69 ± 4.01 | 18.12 ± 5.31 | <0.05 |
| Total ginsenoside capsule | 30 | 12.75 ± 4.03 | 17.91 ± 4.06 | <0.05 |

Efficacy: (shown in Table 19)

TABLE 19
Efficay of three drugs for vascular dementia

| | | | | Markedly | | | | | | |
|---|---|---|---|---|---|---|---|---|---|---|
| | | Cured | | effective | | Effective | | Ineffective | | ORR |
| Drug | n | n | % | n | % | n | % | n | % | % |
| LXN5 capsule | 30 | 2 | 6.7 | 6 | 20.0 | 18 | 60.0 | 4 | 13.3 | 80.7 |
| RSSW capsule | 30 | 0 | 0 | 4 | 13.3 | 18 | 60.0 | 6 | 20.0 | 80.0 |
| Total | 30 | 0 | 0 | 3 | 10.0 | 20 | 66.7 | 7 | 23.3 | 76.7 |
| ginsenoside | | | | | | | | | | |
| capsule | | | | | | | | | | |

(3) Conclusions

LXN5, RSSW capsules, and total ginsenoside capsules had significant improvements in the intelligence of patients with vascular dementia. As shown from Table 21, after 90 patients were treated with LXN5 capsules, RSSW capsules and total ginsenoside capsules for 3 courses, the scale scores of treatment of the three drugs were significantly increased (p<0.01), suggesting that the drugs had the effect of storing memory and improving intelligence. The efficacy of LNX5 capsules was superior to RSSW capsules, and superior to total ginsenoside capsules, suggesting that the compatibility of total ginsenoside, Renshen Shouwu and brain-targeting flavonoids could significantly improve the efficacy for treatment of vascular dementia.

Trial on Efficacy for Patients with Senile Dementia

105 AD patients with the HIS scores<7 points were selected in the trial.

Medication: LNX5 capsules were taken in the morning, noon and evening every day, 2 capsules each time. For Renshen Shouwu (RSSW) capsules and total ginsenoside capsules, 0.3 g per capsule, taken orally, 3 times a day, taken before meals. One course lasted 2 months, 3 courses in total. During the period, other drugs regulating cerebral vasodilation, brain cell metabolism and neurological functions were discontinued.

AD patients were treated by LXN5 capsules, RSSW capsules and total ginsenoside capsules for 3 courses. Results were shown in Table 20.

TABLE 20
Efficacy of two drugs for patients with senile dementia

| | | | | Markedly | | | | | | |
|---|---|---|---|---|---|---|---|---|---|---|
| | | Cured | | effective | | Effective | | Ineffective | | ORR |
| Drug | n | n | % | n | % | n | % | n | % | % |
| LXN5 capsule | 35 | 0 | 0 | 6 | 17.1 | 21 | 60.0 | 8 | 22.9 | 77.1 |
| RSSW capsule | 35 | 0 | 0 | 4 | 11.4 | 18 | 51.4 | 13 | 37.1 | 62.9 |
| Total | 35 | 0 | 0 | 3 | 8.6 | 18 | 51.4 | 14 | 40.0 | 60.0 |
| ginsenoside | | | | | | | | | | |
| capsule | | | | | | | | | | |

As shown from Table 20, the overall response rate of LXN5 capsules for senile dementia was 77.1%, the markedly effective rate was 17.1%, slightly higher than the ORR of current commonly used drugs (70%), and no obvious side effects were observed.

Conclusion: This product could improve the intelligence decline caused by senile dementia. As shown from Table 20, 35 patients had significantly improved their scale scores after taking LXN5 capsules for 2 courses (p<0.01), suggesting that they could restore memory, improve the intelligence, and significantly improve the symptoms such as unsmooth speech, insomnia, irritability, dysphoria, tongue stasis, with an effective rate of 77.1% for 27 cases of AD patients with phlegm blocking and qi stagnation. Its efficacy was higher than the oral RenSen ShouWu (RSSW) capsules, particularly effective to the symptoms such as insomnia, irritability, dysphoria, etc.

The clinical trial results showed that, this product had significant effect for anti-cerebral ischemic injury and improving the learning and memory. It has exact clinical efficacy for the treatment of cerebral ischemic injury related stroke sequelae and vascular dementia and senile dementia. When flavonoids were combined with the brain-targeting formulations, the efficacy of treatment was significantly improved than that without combined with brain-targeting formulations.

### Example 6

LXN6: commercially available rutin (troxerutin or kaempferol extract (content≧60%), vinpocetine (content≧98%), composed at the mass ratio of 1:5. It is made into tablets, 5 mg/tablet.

LXN7: It is made by commercially available troxerutin (content≧98%) and edaravone according to the mass ratio of 1:3.5.

LXN8: It is made into capsule by the commercially available myricetin (content≧95%) and oxiracetam (content≧90%) according to the mass ratio of 1:2.75.400 mg/granule

### Pharmacodynamic Experiment:

Treatment Group: LXN6, LXN7, LXN8 were made into suspensions respectively. Five SD rats were administered the suspensions at the dose of 100 mg/kg and the volume of 20 ml/kg by intragastric administration;

Control group: vinpocetine, edaravone, oxiracetam were made into suspension. Five SD rats were administered the suspension at the dose of 100 mg/kg and the volume of 20 ml/kg by intragastric administration;

The rats were drewd blood 0.5 h after administration, then sacrificed under anaesthesia. Brain tissues were taken to prepare brain homogenates. The content of active ingredient in the blood and brain tissues was detected by UPLC-MS.

In addition, 70 rats of MCAO forebrain ischemia were prepared, and they were administrated with solvent saline, or administrated with LXN6, LXN7 or LXN8, containing vinpocetine, edaravone or oxiracetam, respectively, at a dosage of 100 mg vinpocetine/edaravone/oxiracetam per kg body weight, or administrated with vinpocetine, edaravone or oxiracetam at a dosage of 100 mg/kg.

Intragastric administration at an amount of 20 ml/kg was conducted, for 10 rats per group; Animals were sacrificed 7 days after administration, to observe the improvement rate of cerebral infarction area and the improvement rate of nerve regeneration, learning and memory ability.

The results were shown in Tables 21˜23:

TABLE 21
The active ingredient content (μg/ml) in the blood 0.5 h after administered different
compositions

| | group | | | | | |
|---|---|---|---|---|---|---|
| composition | Vinpocetine | LXN6 | Edaravone | LXN7 | Oxiracetam | LXN8 |
| Vinpocetine | 84.4 | 69.42 | | | | |
| Edaravone | | | 58.31 | 51.1 | | |
| Oxiracetam | | | | | 82.3 | 74.2 |

TABLE 22
The active ingredient content (ng/ml) in the brain tissue 0.5 h after administered
different compositions

| | group | | | | | |
|---|---|---|---|---|---|---|
| composition | Vinpocetine | LXN6 | Edaravone | LXN7 | Oxiracetam | LXN8 |
| Vinpocetine | 124.4 | 369.2 | | | | |
| Edaravone | | | 128.3 | 451.1 | | |
| Oxiracetam | | | | | 172.6 | 761.2 |

The results in Tables 21 an 22 showed that, when different compositions of equal dose of vinpocetine, edaravone, oxiracetam were administered, the contents of vinpocetine, edaravone, oxiracetam had no significant difference in the blood, but when combined with flavonols, the contents of vinpocetine, edaravone, oxiracetam in brain tissues dramatically increased by 3-4 times.

TABLE 23
Protective effect of different compositions on cerebral ischemia 7 days after the
same administration in rats

| Test Item | vinpocetine | LXN6 | edaravone | LXN7 | oxiracetam | LXN8 |
|---|---|---|---|---|---|---|
| Infarction area (%) | 23.1 | 20.4 | 24.1 | 18.1 | 24.5 | 21.1 |
| Nerve regeneration | 87 | 191 | 80 | 192.2 | 85 | 186 |
| rate (%) | | | | | | |
| Improvement rate | 59.9 | 84.5 | 45.1 | 78.5 | 56.9 | 83.5 |
| of learning and | | | | | | |
| memory | | | | | | |
| Space exploration | 56.2 | 74.2 | 51.0 | 76.1 | 54.0 | 75.2 |
| distance (cm) | | | | | | |

The pharmacological evaluation showed that, 7 days after administered LXN6, LXN7 and LXN8, the infarct area was reduced, the cerebral nerve regeneration rate was increased, the learning and memory improvement rate was increased and the space exploration distance (cm) were extended, and the drug efficacy was stronger than vinpocetine, edaravone or oxiracetam alone. Flavonols compounds could promote the TCM active ingredient to pass through the blood-brain barrier, enhance the anti-cerebral ischemic injury and nerve protection, and improve learning and memory function.

### Effects on Vascular Dementia

(1) Clinical Data and Methods

Conditions of Cases: All 128 cases were the inpatients of the Department of Neurology, including 72 males and 54 females; aged 58-76 years, 63.9 years on average; the shortest duration of 1 year, longest duration of 10 years, 3.5 years on average. After clinical examination and neurological scale test, these patients were diagnosed by skull CT or MR.

Diagnostic criteria: The diagnostic criteria for vascular dementia in DSM-IV.

Diagnostic criteria: The diagnostic criteria for vascular dementia in DSM-IV.

Exclusion criteria: Patients with serious neurological, blood, endocrine and other primary diseases, and patients of senile dementia (the score of Hijinski Ischemic Index (HIS)<7, total scores of 18).

Selection of scales: 1. American Simple Intelligence Scale, a total of 30 points, if the score<16 points, it is disturbance of intelligence; 2. Japan Hasegawa Dementia Scale, a total score of 30 points, if the score<16 points, it is dementia; 3. Hijinski Ischemic Index (HIS), a total score of 18 points, if the score>7 points, it is vascular dementia, and if the score<7 points, it is senile dementia.

Efficacy criteria: Comprehensive assessment method was adopted. The changes in the intelligent state before and after treatment were comprehensively assessed, and the change of intelligence was the focus. For cured patients, the score of Hasegawa Dementia Scale was increased to the normal value, for markedly effective, the score was increased by more than 5 points, for effective, the score was increased by less than 5 points, and for ineffective, the score was decreased rather than increased.

### Medication:

LXN6 tablets (5 mg/tablet) and vinpocetine tablets were taken in the morning, noon and evening every day, 1 tablet each time. For oxiracetam and LXN8 capsules, 0.4 g per capsule, taken orally, 2 capsules per time, 3 times a day, taken before meals. One course lasted 2 months, 3 courses in total. During the period, other drugs regulating cerebral vasodilation, brain cell metabolism and neurological functions were discontinued.

(2) Efficacy and Results

The scale scores before and after treatment: (see Tables 24-26)

TABLE 24
Simple intelligent scale scores before and after treatment (x̅ ± s)

| Drug | n | Before treatment | After treatment | P |
|---|---|---|---|---|
| LXN6 tablet | 32 | 12.61 ± 5.23 | 21.63 ± 6.23 | <0.01 |
| Vinpocetine tablet | 32 | 12.63 ± 5.11 | 19.98 ± 6.08 | <0.05 |
| LXN8 capsule | 32 | 13.16 ± 5.31 | 21.26 ± 5.26 | <0.01 |
| Oxiracetam capsule | 32 | 13.03 ± 5.32 | 19.95 ± 5.06 | <0.05 |

TABLE 25
Hasegawa Dementia Scale Scores before and after treatment (x̅ ± s)

| Drug | n | Before treatment | After treatment | P |
|---|---|---|---|---|
| LXN6 tablet | 32 | 12.65 ± 5.66 | 20.86 ± 6.01 | <0.01 |
| Vinpocetine tablet | 32 | 12.61 ± 5.72 | 19.12 ± 5.31 | <0.05 |
| LXN8 capsule | 32 | 12.45 ± 5.15 | 20.76 ± 5.77 | <0.01 |
| Oxiracetam capsule | 32 | 12.41 ± 5.21 | 19.04 ± 5.23 | <0.05 |

Efficacy: (shown in Table 26)

TABLE 26
Efficacy of three drugs for vascular dementia

| | | | | Markedly | | | | | | |
|---|---|---|---|---|---|---|---|---|---|---|
| | | Cured | | effective | | Effective | | Ineffective | | ORR |
| Drug | n | n | % | n | % | n | % | n | % | % |
| LXN6 tablet | 32 | 2 | 6.2 | 8 | 25.0 | 18 | 56.3 | 4 | 12.5 | 87.5 |
| Vinpocetine | 32 | 0 | 0 | 8 | 25.0 | 18 | 56.3 | 6 | 18.8 | 81.2 |
| tablet | | | | | | | | | | |
| LXN8 | 32 | 3 | 9.4 | 8 | 25.0 | 18 | 56.3 | 3 | 9.4 | 90.6 |
| capsule | | | | | | | | | | |
| Oxiracetam | 32 | 1 | 3.1 | 6 | 18.8 | 20 | 62.5 | 5 | 15.6 | 84.4 |
| capsule | | | | | | | | | | |

(3) Conclusions

LXN6 tablets, vinpocetine tablets, LXN8 capsules and oxiracetam capsules had significant improvements in the intelligence of patients with vascular dementia. As shown from Table 6-6, after 128 patients were treated with LXN6 tablets, vinpocetine tablets, LXN8 capsules and oxiracetam capsules for 3 courses, the scale scores of treatment of the four drugs were significantly increased (p<0.01), suggesting that the drugs had the effect of storing memory and improving intelligence. The efficacy of LXN6 tablets was superior to vinpocetine tablets, and that of LXN8 capsules was superior to oxiracetam capsules, suggesting that the brain-targeting compatibility significantly improved the efficacy of the drugs for treatment of vascular dementia.

### Example 7

LXN9: composed by the commercially available kaempferol extract (content 60%) and levodopa at a mass ratio of 1:4.0.

LXN10: composed by Hesperidin extract (content 96%) and levodopa at a mass ratio of 1:3.3.

### Pharmacodynamic Experiment:

Treatment Group: LXN9 and LXN10 were made into suspension. Five SD rats were administered the suspension at the dose of 100 mg/kg levodopa and the volume of 20 ml/kg by intragastric administration;

Control group: The levodopa was made into suspension. Five SD rats were administered the suspension at the dose of 100 mg/kg and the volume of 20 ml/kg by intragastric administration;

The rats were drew blood 0.5 h after administration, and then sacrificed under anaesthesia. Brain tissues were taken to prepare brain homogenates. The content of active ingredient in the blood and brain tissues was detected by UPLC-MS.

In addition, 40 rats of 6-hydroxydopamine PD models were prepared, and they were administered solvent saline, LXN9, LXN10 and levodopa according to the equal dose of 100 mg/kg levodopa and the volume of 20 ml/kg by gavage, 10 rats per group; Animals were administered for 15 days, to observe the changes of five indexes, including slow-moving test, grasping experiment, tail stiffness, change of tonic symptoms and tremor test. The test results were shown in tables 27-28:

TABLE 27
The levodopa content in the blood and brain tissues 0.5 h
after administered different compositions

| Group | Levodopa | LXN9 | LXN10 |
|---|---|---|---|
| Content in the blood (μg/ml) | 114.2 | 99.4 | 97.6 |
| Content in the brain tissues (ng/ml) | 284.4 | 869.4 | 841.3 |

Table 27 showed that, when administered different compositions with equal dose of levodopa, the content of levodopa in the blood showed no significant difference, but when combined with the flavonols, the content of levodopa in the brain tissues was increased by 3 times dramatically.

TABLE 28
Protective effect of different compositions on cerebral ischemia
7 days after administration in rats

| | Model | | | |
|---|---|---|---|---|
| Test Item | group | Levodopa | LXN9 | LXN10 |
| Duration of slow-moving | 38.5 | 15.5 | 7.5 | 5.8 |
| test (minutes) | | | | |
| Grip Test Duration (min) | 61.8 | 40.1 | 35.6 | 34.8 |
| tail stiffness time (min) | 36.3 | 10.2 | 7.4 | 5.3 |
| Muscle tremor frequency | 53.2 | 10.1 | 8.4 | 7.6 |
| (times, minutes) | | | | |
| EMG group discharge | 7.5 | 3.1 | 2.6 | 2.7 |
| frequency (times/sec) | | | | |

The pharmacodynamic evaluation showed that, after administered LXN9 and LXN10 for 15 days, the symptoms of delayed action, grasping and tail stiffness were significantly improved, the muscle tremor frequency and EMG group discharge frequency were significantly reduced. The efficacy was stronger than levodopa alone. Flavonol compounds could promote levodopa to pass through the blood-brain barrier and enhance its pharmacological effects on Parkinson's disease.

### Treatment Effect of LXN9, LXN10 for Parkinson's Disease

(1) Clinical Data and Methods
Same as above.

(2) Sources of Patients: Outpatients and Inpatients from First Affiliated Hospital of Guangdong College of Pharmacy.

Profile of patients: All selected patients were from department of neurology, which diagnosed with Parkinson's disease according to the Parkinson's disease diagnostic criteria in clinical examination during 2010-2014. a total of 120 cases, including 66 males, 54 females; aged 50-78 years, and 62.8 years on average, with the shortest duration of 1 year, and longest duration of 13 years, 3.6 years on average.

All eligible cases were randomly divided into madopar treatment group, LXN9 group and LXN 10 group, of which,

Levodopa treatment group: 40 cases, including 22 males and 18 females; duration of 1-13 years, 3.6 years on average; aged 50-78 years, 62.4 years on average.

LXN9 capsule group: 40 cases, including 22 males and 18 females; duration of 1-12 years, 3.7 years on average; aged 51-76 years, 62.2 years on average.

LXN10 capsule group: 40 cases, including 22 males and 18 females; duration of 1-13 years, 3.6 years on average; aged 50-77 years, 61.5 years on average.

By statistical analysis of the above 3 groups of patients, the differences in the gender, duration and age were not statistically significant (P>0.05) and were comparable. All subjects were informed and signed the informed consent.

(3) Treatment Regimens

Levodopa tablet group: (specification of 250 mg/tablet) 1 tablet/time, 3 times/d.

LXN9 capsule group: 250 mg/capsule, 1 capsule per time, 3 times a day.

LXN 10 capsule group: 250 mg/capsule, 1 capsule per time, 3 times a day.

Fifteen days as a course in all groups, a total of 9 months for medication; and during the period, other drugs regulating cerebral vasodilation, brain cell metabolism and neurological functions were discontinued.

In all groups, after treatment for 1 month, 3 months, 6 months and 9 months, their curative effects were evaluated.

(4) Observation Indexes

Same as above.

(5) Statistical Method

Same as above.

(6) Analysis of Curative Effect

Table 29 showed that the drugs in the invention could effectively improve the main symptoms and signs of Parkinson's disease. According to the recognized efficacy standard, the overall treatment of Parkinson's disease had achieve good clinical efficacy; and according to the comparison between groups, the efficacy of LXN9 was equivalent to LXN10, and superior to Levodopa capsules, with statistically significant difference (p<0.01).

TABLE 29
Curative effect for PD in the three groups

| | | | | | | | | | | Overall |
|---|---|---|---|---|---|---|---|---|---|---|
| | Number | Clinically | | Markedly | | | | | | response |
| | of cases | cured | | effective | | Effective | | Ineffective | | rate (ORR) |
| Group | (n) | n | % | n | % | n | % | n | % | % |
| LXN9 capsule | 40 | 3 | 7.8 | 20 | 50.0 | 14 | 35.0 | 3 | 7.8 | 92.2 |
| LXN10 capsule | 40 | 3 | 7.8 | 19 | 47.8 | 16 | 40.0 | 2 | 5.0 | 95.0 |
| Levodopa | 40 | 0 | 0 | 19 | 47.8 | 13 | 32.2 | 8 | 20.0 | 80.0 |

Compared with Levodopa,
p < 0.05.

The clinically cured, markedly effective and effective cases were used as the basis for calculating the overall response rate. For comparison of clinical efficacy in the three groups, the ORRs of the LXN9 capsule treatment group and LXN10 capsule treatment group were 92.2% and 95.0, respectively, and the ORR of Levodopa control group 2 was 80%. The comparison among the three groups showed significant difference (p<0.05).

A. Psychiatric, behavioral, and emotional scores (UPDRS-1): After treatment, the psychiatric, behavioral, and emotional scores of the LXN9, LXN10 groups improved significantly. The efficacy of LXN9 capsules was equivalent to LXN10 capsules, which was superior to RSSW capsules and Levodopa, showing statistically significant difference (p<0.05), as shown in Table 30.

B. Daily Living Activity (UPDRS-II) Score: After treatment, the efficacy of LXN9 capsules was equivalent to LXN10 capsules, and superior to RSSW capsules and Levodopa, showing statistically significant difference (p<0.05), as shown in Table 31.

C. Motor function examination (UPDRS-III) score: After treatment, the scores of various motor function examinations were significantly improved, and the efficacy of LXN9 and LXN10 capsules was superior to RSSW capsules and total ginsenoside capsules, showing statistically significant difference (p<0.05), as shown in Table 32.

D. Motor complications (UPDRS-IV) scores: After treatment, the motor complications scores in various groups were significantly improved compared with the control group 2. The efficacy of LXN9 capsules was equivalent to LXN10 capsules, which was superior to RSSW capsules and Levodopa, showing statistically significant difference (p<0.05), as shown in Table 33.

E. Scores of Quality of Life Scale (PDQ): After treatment, the scores of quality of life scale in various groups were significantly improved compared with the control group 2. The efficacy of LXN9 capsules was equivalent to LXN10 capsules, which was superior to RSSW capsules and Levodopa, showing statistically significant difference (p<0.05), as shown in Table 34.

TABLE 30
Changes of psychiatric, behavioral, and emotional scores (UPDRS-1) before and
after treatment (x̅ ± s)

| | | | 1 month | 3 months | 6 months | |
|---|---|---|---|---|---|---|
| | | Before | after | after | after | 12 months |
| Group | n | treatment | treatment | treatment | treatment | after treatment |
| LXN9 | 40 | 3.66 ± 1.01 | 2.79 ± 0.65 | 1.53 ± 0.56 | 1.16 ± 0.52 | 1.02 ± 0.61** |
| capsule | | | | | | |
| LXN10 | 40 | 3.63 ± 0.99 | 3.06 ± 0.73 | 1.98 ± 0.54 | 1.69 ± 0.54 | 1.33 ± 0.63** |
| capsule | | | | | | |
| Human | 40 | 3.59 ± 1.02 | 3.15 ± 0.81 | 2.29 ± 0.73 | 1.85 ± 0.53 | 1.79 ± 0.59 |
| Levodopa | | | | | | |

Note:
*compared with total ginsenoside capsule, p < 0.05,
**p < 0.01.

TABLE 31
Changes of Daily Living Activity (UPDRS-II) Score before and after treatment
(x̅ ± s)

| | | | 1 month | 3 months | 6 months | 12 months | |
|---|---|---|---|---|---|---|---|
| | | Before | after | after | after | after | |
| Group | n | treatment | treatment | treatment | treatment | treatment | p |
| LXN9 | 40 | 15.65 ± 5.66 | 13.86 ± 5.27 | 11.16 ± 5.03 | 8.86 ± 4.27* | 6.16 ± 3.51** | <0.05 |
| capsule | | | | | | | |
| LXN10 | 40 | 15.63 ± 5.69 | 13.58 ± 5.13 | 11.28 ± 4.87 | 9.08 ± 4.13* | 5.89 ± 3.66* | <0.05 |
| capsule | | | | | | | |
| Human | 40 | 15.61 ± 5.71 | 14.12 ± 5.31 | 12.49 ± 5.10 | 11.12 ± 3.31 | 10.32 ± 3.31 | <0.05 |
| Levodopa | | | | | | | |

TABLE 32
Changes of motor function examination (UPDRS-III) scores before and after
treatment (x̅ ± s)

| | | | 1 month | 3 months | 6 months | 12 months |
|---|---|---|---|---|---|---|
| | | Before | after | after | after | after |
| Group | n | treatment | treatment | treatment | treatment | treatment |
| LXN9 | 40 | 38.61 ± 9.01 | 31.96 ± 8.18 | 20.73 ± 6.52* | 16.63 ± 6.13* | 13.13 ± 6.13* |
| capsule | | | | | | |
| LXN10 | 40 | 38.16 ± 9.01 | 31.16 ± 8.23 | 20.71 ± 6.54* | 16.15 ± 6.21 | 12.98 ± 6.31 |
| capsule | | | | | | |
| Levodopa | 40 | 37.99 ± 10.23 | 34.16 ± 7.91 | 28.91 ± 6.61 | 24.85 ± 6.56 | 21.15 ± 6.56 |

TABLE 33
Changes of motor complications (UPDRS-IV) scores before and after treatment
(x̅ ± s)

| | | | 1 month | 3 months | | 12 months |
|---|---|---|---|---|---|---|
| | | Before | after | after | 6 months after | after |
| Group | n | treatment | treatment | treatment | treatment | treatment |
| LXN9 | 40 | 3.06 ± 1.01 | 2.29 ± 0.83 | 1.68 ± 0.62* | 1.29 ± 0.63** | 1.23 ± 0.53* |
| capsule | | | | | | |
| LXN10 | 40 | 3.06 ± 1.01 | 2.31 ± 0.81 | 1.71 ± 0.63 | 1.31 ± 0.51* | 1.21 ± 0.52* |
| capsule | | | | | | |
| Levodopa | 40 | 3.59 ± 1.02 | 2.93 ± 0.86 | 2.46 ± 0.61 | 2.15 ± 0.56 | 1.79 ± 0.56 |

TABLE 34
Changes of daily life satisfaction (LSIB) scores before and after treatment (x̅ ± s)

| | | Before | 1 month after | 3 months after | 6 months after | 12 months |
|---|---|---|---|---|---|---|
| Group | n | treatment | treatment | treatment | treatment | after treatment |
| LXN9 | 40 | 12.66 ± 2.01 | 14.64 ± 3.81 | 16.79 ± 3.65 | 18.63 ± 3.18 | 20.33 ± 3.43 |
| capsule | | | | | | |
| LXN10 | 40 | 12.48 ± 2.11 | 14.83 ± 3.48 | 17.14 ± 3.51 | 18.58 ± 3.31 | 20.15 ± 3.31 |
| capsule | | | | | | |
| Levodopa | 40 | 12.59 ± 2.02 | 13.11 ± 3.92 | 14.91 ± 3.16 | 16.85 ± 3.15 | 17.24 ± 3.16 |

The drugs in the invention have a markedly ameliorating effect on dyskinesias of Parkinson's disease (PD). As shown from tables 29-34, after 120 patients were treated with LXN9 capsules, LXN10 capsules, and Levodopa for 24 courses, the treatment scale scores of the three groups of drugs were significantly increased (p<0.01), indicating that the drugs had the effect of relieving muscle tremor and muscle stiffness, and dramatically improving the symptoms such as unsmooth speech, insomnia, irritability, dysphoria, tongue stasis. The effective rates of LXN9 capsules, LXN10 capsules, and Levodopa on treatment of 40 patients with Parkinson's disease (PD) were 92.2%, 95% and 80.0% respectively, although the cure rate was about 7.8% and the markedly effective rates were 50%, 47.8% and 47.8% respectively. The efficacy of the brain-targeting drug in the treatment of PD was higher than 80.0%, the overall response rate of conventional levodopa tablets, showing significant difference (p<0.05).

Several embodiments are employed to explain the principles and mode of execution of the invention. These embodiments are only used for assistance in understanding the methods and core concepts of the invention; in addition, to those skilled in the art, it should be understood that the invention is not limited to the details of the invention, but rather should be construed as embodying the principles and the scope of the application of the invention.

1. A method for promoting drugs that have therapeutic or health effects on brain diseases to pass through the blood-brain barrier in a subject, comprising administering to a subject in need thereof an effective amount of said drug(s) and flavonol.

2. The method according to claim 1, wherein the flavonol is selected from kaempferide, myricetin, hesperidin, rutin, troxerutin, and hydroxy derivatives thereof.

3. The method according to claim 2, wherein the hydroxy derivatives of kaempferol, myricetin, hesperidin, rutin or troxerutin are of their glycosides, esters and ethers.

4. The method according to claim 1, wherein the drug having a therapeutic or healthcare effect on the brain disease is selected from ginsenoside, stilbene glucoside of Radix polygoni multiflori, resveratrol, levodopa, edaravone, vinpocetine, nicergoline, citicoline, and oxiracetam.

5. A composition, wherein its active ingredients are synergist flavonol and active molecule(s) having a therapeutic or healthcare effect on brain diseases.

6. The composition according to claim 5, wherein the flavonol is selected from kaempferide, myricetin, hesperidin, rutin, troxerutin, and hydroxy derivatives thereof.

7. The composition according to claim 5, wherein the drug having a therapeutic or healthcare effect on brain diseases is selected from ginsenoside, stilbene glucoside of Radix polygoni multiflori, resveratrol, levodopa, edaravone, vinpocetine, nicergoline, citicoline, and oxiracetam.

8. The composition according to claim 7, wherein the active ingredients of the composition are:
kaempferide, total ginsenosides extract and stilbene glucoside at a mass ratio of 1:1˜5:1˜5, or,
troxerutin, total ginsenosides extract and stilbene glucoside at a mass ratio of 1:1˜5:1˜2, or,
at least one of rutin and troxerutin, and vinpocetine; wherein the mass ratio of the sum of rutin and troxerutin to vinpocetine is 1:2˜5;
or myricetin and levodopa at a mass ratio of 1:2˜5;
or troxerutin and levodopa at a mass ratio of 1:2˜5;
or hesperidin and levodopa at a mass ratio of 1:1.5˜5;
or kaempferol and oxiracetam at a mass ratio of 1:2˜5;
or troxerutin and edaravone at a mass ratio of 1:2˜5;
or troxerutin and citicoline at a mass ratio of 1:2˜5.

9. The composition according to claim 5, wherein the brain disease is selected from a cerebral ischemic injury disease and a neurodegenerative disease.

10. The composition according to claim 9, wherein the brain disease is selected from ischemic stroke, stroke sequelae, vascular dementia, senile dementia, and Parkinson's disease.
